(19) United States
(12) Patent Application Publication
(10) Pub. No.: US 2007/0027190 A1
(43) Pub. Date: Feb. 1, 2007

(54) Antibacterial fab i inhibitors

(76) Inventors: Donald T. Moir, Lexington, MA (US); Yibin Xiang, Acton, MA (US); Anthony C. Arvanites, New Bedford, MA (US); Syed Masarrat Ali, North Andover, MA (US); Bolin Geng, Andover, MA (US); Mark A. Ashwell, Carlisle, MA (US); Hernan Antonio Orgueira, Cambridge, MA (US); Alan P. Kaplan, Kings Park, NY (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/542,351

(22) PCT Filed: Jan. 16, 2004
(86) PCT No.: PCT/US04/01327

§ 371 Date: Aug. 7, 2006

Related U.S. Application Data
(60) Provisional application No. 60/441,411, filed on Jan. 17, 2003.

(51) Int. Cl. A61K 31/4439; A61K 31/4436; A61K 31/4412

(52) U.S. Cl. 514/341; 514/342; 514/344

(57) ABSTRACT

Disclosed herein are antibacterial compounds that inhibit fabl, a NADH-dependent enoyl [acyl carrier protein] reductase enzyme in the fatty acid biosynthesis pathway. The compounds are represented by structural formulas Ia and Ib: R1 and R2 are independently monocyclic aryl or heteroaryl groups, wherein the groups represented by R1 and R2 are optionally substituted with one or more acyclic substituents; R3 is —H or an optionally substituted C1-C8 aliphatic, C3-C8 cycloaliphatic, aryl, or heteroaryl group. X1 is a bond or a C1-C3 alkylene chain that is optionally substituted with a C1-C4 alkyl or an acidic group. X2 is an aryl, heteroaryl or C3-C8 cycloaliphatic ring, wherein the group represented by X2 is optionally substituted with triazole, tetrazole, and/or one or more acyclic substituents.

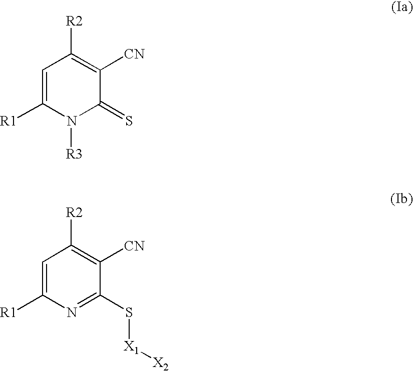

### RELATED APPLICATION

This application claims the benefit of U.S. Provisional Application No. 60/441,411, filed on Jan. 17, 2003. The entire teachings of the above application is incorporated herein by reference.

### BACKGROUND OF THE INVENTION

In the last century, many antibiotics were developed that led to significant reductions in mortality. Unfortunately, extensive use of antibiotics, coupled with natural evolutionary processes has led to bacteria that are resistant to multiple antibiotics. For example, methicillin/oxacillin resistant Staphyloccocus aureus (MRSA), vancomycin resistant enterococci (VRE), and penicillin-resistant Streptococcus pneumoniae (PRSP) are especially problematic. Some bacteria are resistant to a wide range of antibiotics, for example, strains of Mycobacterium tuberculosis exist that are resistant to antibiotics including isoniazid, rifampin; ethambutol, streptomycin, ethionamide, kanamycin, and rifabutin.

In addition, to multidrug resistance, global travel has helped to spread relatively unknown bacteria from isolated areas to new populations. Furthermore, there is the threat of bacteria as biological weapons. These bacteria may not be easily treated with existing antibiotics.

A wide range of infectious bacteria use the fatty acid biosynthesis pathway, and in particular, NADH-dependent enoyl ACP (acyl carrier protein) reductases, or fabI proteins, as the last step in the pathway.

Therefore, there is a need for new antibiotics that target such proteins, whereby bacteria dependent on the pathway may be treated.

### SUMMARY OF THE INVENTION

It has now been found that certain thiol pyridines and pyridothiones have antibiotic properties as inhibitors of fabI, a NADH-dependent enoyl ACP reductase enzyme in the fatty acid biosynthesis pathway, as shown in Example 3. Furthermore, the disclosed compounds are found to have antibiotic activity against drug-resistant bacteria, as shown in Example 4. Based on these discoveries, FabI inhibitors, pharmaceutical compositions comprising the disclosed FabI inhibitors and methods of treatment with the disclosed FabI inhibitors are provided herein.

One embodiment of the invention is a method of treating a subject for a bacterial infection by administering to the subject an effective amount of a compound, or a pharmaceutically acceptable salt thereof, wherein the compound is represented by structural formula Ia:

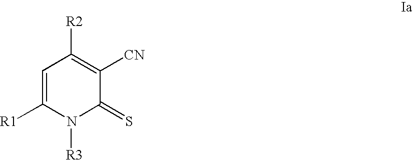

R1 and R2 are independently monocyclic aryl or heteroaryl groups, wherein the groups represented by R1 and R2 are optionally substituted with triazole, tetrazole, or one or more acyclic substituents; provided that R1 is not thienyl when R2 is alkoxy-substituted phenyl. R3 is —H or an optionally substituted C1-C8 aliphatic, C3-C8 cycloaliphatic, aryl, or heteroaryl group. The compound can also be represented by structural formula Ib, or a pharmaceutically acceptable salt thereof:

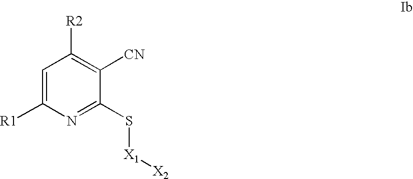

R1 and R2 are independently monocyclic aryl or heteroaryl groups, wherein the groups represented by R1 and R2 are optionally substituted with triazole, tetrazole, or one or more acyclic substituents. X1 is a bond or a C1-C3 alkylene chain that is optionally substituted with a C1-C4 alkyl or an acidic group. X2 is an aryl, heteroaryl or C3-C8 cycloaliphatic ring, wherein the group represented by X2 is optionally substituted with triazole, tetrazole, and/or one or more acyclic substituents. Alternatively, X2 is triazole, tetrazole, carboxyl, —(CO)NR^{a}R^{b}, —(C═NH)NR^{a}R^{b}, or —(CS)NR^{a}R^{b}. R^{a} and R^{b} are independently —H or an optionally substituted group selected from aryl, heteroaryl, C3-C8 cycloaliphatic, and C1-C4 alkyl; or alternatively, R^{a} and R^{b}, taken together with the nitrogen to which they are bonded, are an optionally substituted non-aromatic heterocyclic group.

Another embodiment is a pharmaceutical composition comprising a pharmaceutically acceptable carrier or diluent and a compound represented by structure Ib, wherein R1, R2, and X1 are as defined in the method above, and X2 is an an aryl or heteroaryl ring. An aryl or heteroaryl represented by X2 is optionally substituted with triazole, tetrazole, and/or one or more acyclic substituents, or X2 is triazole, tetrazole, an acidic group, —(CO)NR^{a}R^{b}, —(C═NH)NR^{a}R^{b}, or —(CS)NR^{a}R^{b}, wherein R^{a} and R^{b} are independently —H or an optionally substituted group selected from aryl, heteroaryl, and C1-C4 alkyl; provided that if both R^{a} and R^{b} are, —H, neither R1 not R2 are furanyl or pyridyl. In some aspects, R1 and R2 are not furanyl or pyridyl when X1 is an acidic group.

Another embodiment is a compound represented by structural formula Ib or a pharmaceutically acceptable salt thereof, wherein R1, R2, and X1 are as defined in the method above, and X2 is an aryl or heteroaryl ring, wherein the group represented by X2 is optionally substituted with triazole, tetrazole, and/or one or more acyclic substituents, or X2 is triazole, tetrazole, —(CO)NR^{a}R^{b}, —(C═NH)NR^{a}R^{b}, or —(CS)NR^{a}R^{b}, wherein R^{a} and R^{b} are independently —H or an optionally substituted group selected from aryl, heteroaryl, and C1-C4 alkyl; provided that both R^{a} and R^{b} are not —H. The compound is as defined above, provided that the compound is not represented by one of structural formulas A, B, C, or D (described in the following detailed description).

The invention is useful as a treatment or prophylactic for infections caused by bacteria that depend on the fatty acid biosynthesis pathway, and in particular, bacteria that express a fabI enzyme. Furthermore, it is useful against bacteria that have developed antibiotic resistance, especially multiple drug resistant strains, because it acts through a different mechanism than existing, widely used antibiotics.

### BRIEF DESCRIPTION OF THE DRAWINGS

FIG. 1 is a graph depicting the effect of fabI inhibitor A in Staphyloccocus aureus at various concentrations.

FIG. 2 is a graph depicting the effect of fabI inhibitor A in a control Staphyloccocus aureus strain and a strain that under-expresses fabI.

### DETAILED DESCRIPTION OF THE INVENTION

The invention is generally related to methods, compounds, and pharmaceutical compositions for treating and preventing bacterial infections. In particular, the invention relates to substituted thiopyridines and pyridothiones that are fabI inhibitors.

In alternative embodiments, the variables for the compound represented by Formula Ia-b are as provided above, provided that the compound is further characterized by one or more of the following features.

In a preferred embodiment, R1 and R2 in Ia and Ib are selected from optionally substituted phenyl, pyridyl, pyrazinyl, pyrimidyl, triazinyl, thienyl, furanyl, pyrrolyl, pyrazolyl, thiazolyl, isothiazolyl, oxazolyl, and isoxazolyl, and the remaining variables are as provided above. More preferably, R1 and R2 are independently selected from optionally substituted phenyl, pyridyl, thienyl, furanyl, and pyrrolyl.

Suitable acyclic substituents for aryl and heteroaryl groups represented by R1 and R2 are provided herein below in the section describing substituents for aryl and heteroaryl groups. Preferred substituents for the groups represented by R1 and R2 include triazole, tetrazole, and acyclic substituents including halogen, —OH, —R^{d}, —OR^{d}, carboxyl, sulfate, sulfonate, —NO₂, —NH₂, —NHCOR^{d}, —CONR^{e}₂, —NR^{e}₂, or —SO₂NH₂. Each R^{d} is independently a C1-C4 alkyl optionally substituted with 1, 2, or 3 halogens, for example, —CF₃. Each R^{e} is an independently selected C1-C4 alkyl, or both R^{e} groups, taken together with the nitrogen atom to which they are bonded, are a 4 to 7 membered non-aromatic heterocyclic group. For example, —NR^{e}₂ can be dimethylamine, ispropylamine, di-tert-butylamine, N-piperidine, and the like. More preferred substituents for the groups represented by R1 and R2 include halogen, —R^{d}, —OR^{d}, or —NO₂.

More preferably, at least one of the groups represented by R1 and R2 is a phenyl group substituted with —F, —Cl, —NO₂, or —OCH₃. In a particularly preferred alternative, R2 is a phenyl substituted with —F, —Cl, —NO₂, or —OCH₃ and R1 is 2-thienyl.

Even more preferably, R1 is selected from structural formulas R1^{a}-R1^{c}:

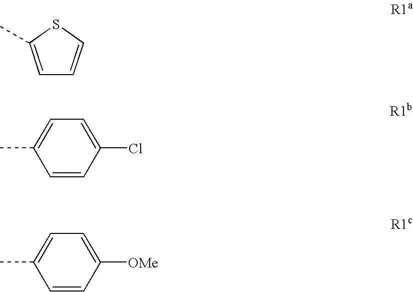

and R2 can be represented by structural formulas R2^{a}-R2^{e}:

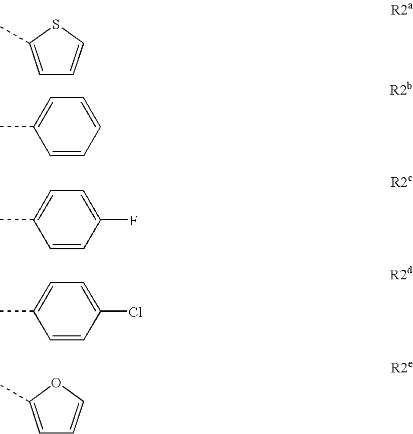

In a preferred embodiment, the disclosed fabI inhibitor is represented by structural formula Ib, R1 and R2 are as described in the preceding four paragraphs, X1 is a bond or a C1-C3 alkylene chain that is optionally substituted with C1-C4 alkyl, triazole, tetrazole, carboxyl, sulfate, sulfonate, and X2 is —(CO)NR^{a}R^{b} or an optionally substituted aryl or heteroaryl group. Preferred values for X2 include an optionally substituted phenyl, pyridyl, thienyl, furanyl, or pyrrolyl. More preferably, X2 is a phenyl group substituted with halogen, —R^{d}, —OR^{d}, —NHCOR^{d}, —CONR^{e}₂, triazole, tetrazole, carboxyl, —CH₂COOH, —CH₂CH₂COOH, —NO₂, sulfate, or sulfonate. Alternatively, X1 is a C1-C2 alkylene group optionally substituted with methyl and X2 is a phenyl substituted with a triazole, tetrazole, —CH₂CO₂H, —CH₂CH₂CO₂H, carboxyl, or —NHCOCH₃, and optionally one or more groups selected from halogen, —R^{d}, —OR^{d}, —NO₂, sulfate, and sulfonate. More preferably, X2 is an unsubstituted phenyl or a phenyl substituted with —CH₂CO₂H, —CH₂CH₂CO₂H, carboxyl, or —NHCOCH₃. Each R^{d} and R^{e} are as described above and are independently selected.

In another preferred embodiment, the disclosed fabI inhibitor is represented by structural formula Ib, R1 and R2 are as described in the preceding five paragraphs, X1 is a C1-C2 alkylene chain substituted with triazole, tetrazole, or carboxyl, while X2 is a phenyl or heteroaryl group optionally substituted with halogen, —R^{d}, —OR^{d}, —NHCOR^{d}, —CONR^{e}₂, triazole, tetrazole, carboxyl, —NO₂, sulfate, or sulfonate. Preferably, X2 is an unsubstituted phenyl or heteroaryl group, or is substituted with carboxyl, and R1 and R2 have the values described in the preceding paragraph. Each R^{d} and R^{e} are as described above and are independently selected.

In a more preferred embodiment, the compound is represented by structural formula IIb:

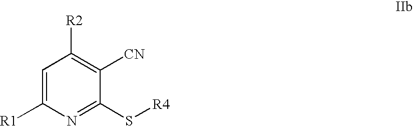

R1 represented by a structural formula selected from R1^{a} to R1^{c}, R2 is represented by a structural formula selected from R2^{a} to R2^{e}, and R4 is selected from the groups represented by structural formulas R4^{a}-R4^{l}, preferably R4^{a}-R4^{j}, and more preferably R4^{a}-R4^{f}:

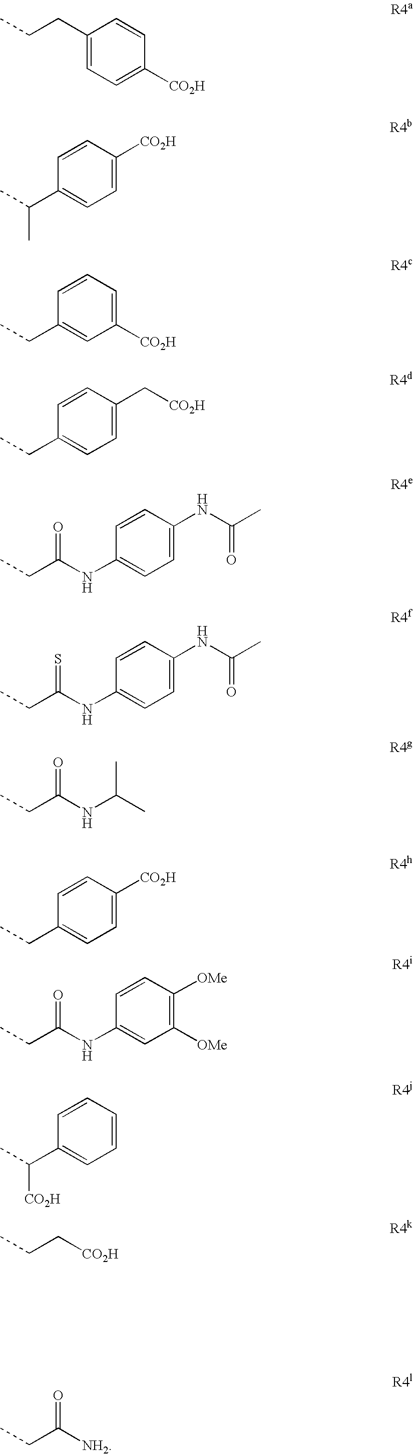

In still another preferred alternative, R1 in structural formula IIb is the group represented by structural formula R1^{a}. In yet another preferred alternative, R2 in structural formula IIb is the group represented by structural formula R2^{a}. In another preferred embodiment, R4 in structural formula IIb is selected from the groups represented by structural formulas R4^{a}-R4^{e}. In another preferred embodiment, R1 in structural formula IIb is the group represented by structural formula R1^{c}, R2 is selected from group represented by structural formulas R2^{b}, R2^{c}, and R2^{e}; and R4 in structural formula IIb is selected from the groups represented by structural formulas R4^{e} and R4^{g}.

Specific examples of the disclosed fabI inhibitors are represented below by structural formulas A-O.

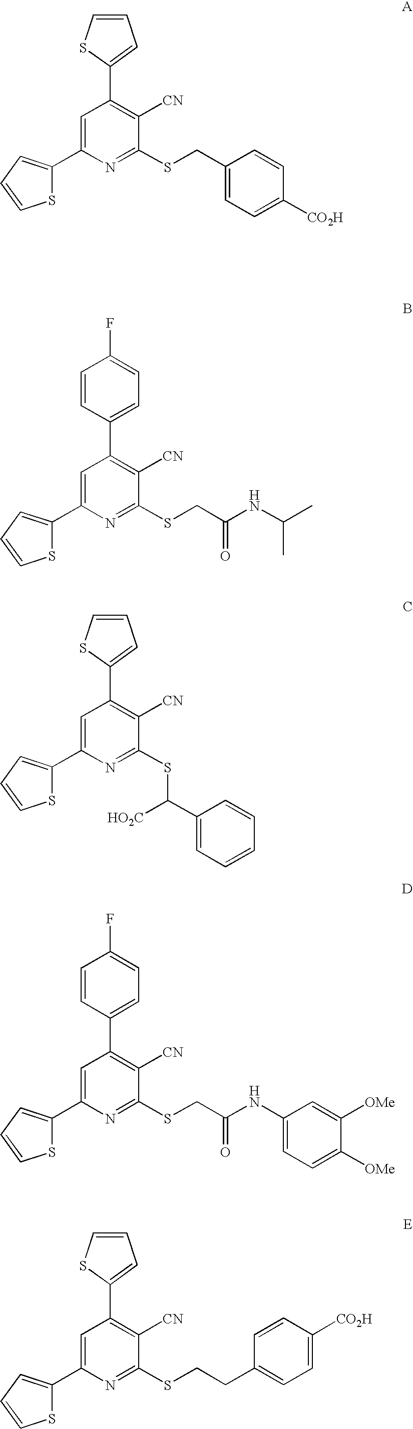

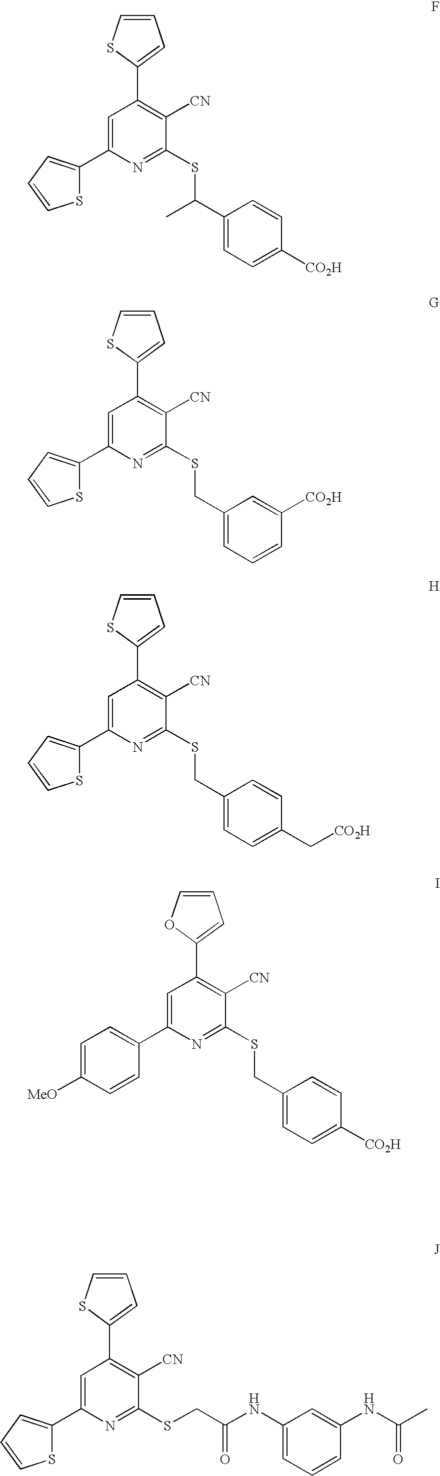

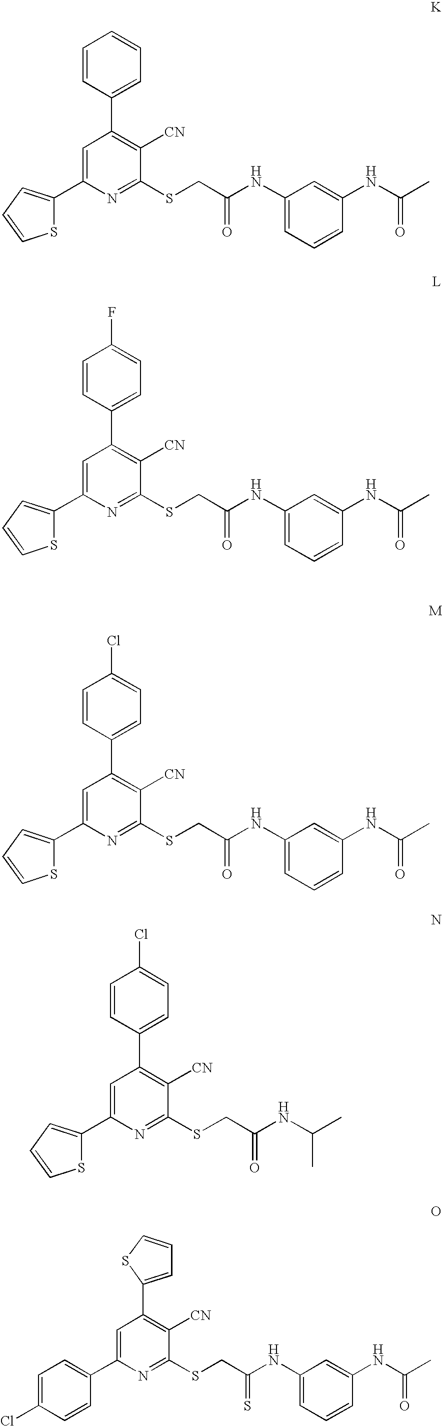

In another preferred embodiment, the fabI inhibitor of the present invention is represented by structural formula Ic:

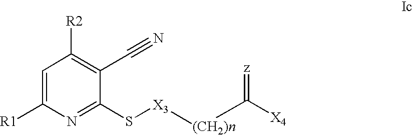

R1 and R2 are as described for structural formula Ib. Z is O, S or NR^{f}, X3 is: i) a bond; ii) a C1-C3 alkylene chain that is optionally substituted with a C1-C4 alkyl group or an aromatic group (preferably a phenyl group); or iii) a group represented by: 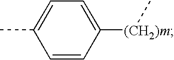
n and m are independently 0 or 1; X4 is —OH or —NR^{g}R^{h}; R^{f} is H or a C1-C4 alkyl group; and R^{g} and R^{h} are independently —H or an optionally substituted group selected from: i) aryl that is optionally substituted with one or two C1-C4 alkyl groups, alkoxy groups or acetamido groups; ii) heteroaryl; iii) C3-C8 cycloaliphatic or C1-C6 straight or branched alkyl. Pharmaceutically acceptable salts of the compound represented by structural formula Ic are also included. In some aspects, Compound A-D are excluded from structural formula Ic.

Preferably for structural formula Ic, R1 and R2 have one of the preferred values as described for structural formula Ib. More preferably, Z is O; and X4 is OH.

The present invention also includes pharmaceutical compositions comprising a pharmaceutically acceptable carrier or diluent and a compound represented by structural formula Ic (or a pharmaceutically acceptable salt thereof) and a method of treating a subject for a bacterial infection by administering to the subject an effective amount of a compound represented by structural formula Ic (or a pharmaceutically acceptable salt thereof).

In the structural formulas depicted herein, a dashed line indicates a bond by which the depicted or moiety or group is connected to the remainder of the molecule. For example, the dashed line in R1^{a} indicates the bond that connects the depicted thienyl group to the pyridylthione ring of Ia, the pyridyl ring of Ib or IIb.

A “subject” includes mammals, e.g., humans, companion animals (e.g., dogs, cats, and the like), farm animals (e.g., cows, sheep, pigs, horses, and the like) and laboratory animals (e.g., rats, mice, guinea pigs, and the like). In a preferred embodiment of the disclosed method, the subject is human.

A subject in need of treatment has a bacterial infection or has been exposed to an infectious bacterium, the symptoms of which may be alleviated by administering an effective amount of the disclosed fabI inhibitors. For example, a subject in need of treatment can have an infection for which the disclosed fabI inhibitors can be administered as a treatment. In another example, a subject in need of treatment can have an open wound or burn injury, for which the disclosed compounds can be administered as a prophylactic. Typically, a subject will be treated for an existing bacterial infection. A subject can have a bacterial infection caused by Acinetobacter baumanii, Bacillus anthracis, Citrobacter sp., Escherichia coli, Enterobacter sp., Enterococcus faecalis, Enterococcus faecium, Francisella tularensis, Haemophilus influenzae, Klebsiella sp., Listeria monocytogenes, Moraxella catarrhalis, Mycobacterium tuberculosis, Neisseria meningitidis, Proteus mirabilis, Proteus vulgaris, Pseudomonas aeruginosa, Salmonella sp., Serratia sp., Shigella sp., Stenotrophomonas maltophilia, Staphylococcus aureus, or Staphylococcus epidermidis. Preferably, the subject is treated (prophylactically or therapeutically) for a bacterial infection caused by a bacterium that expresses a fabI protein. As used herein, a fabI protein is a NADH-dependent enoyl ACP (acyl carrier-protein) reductase enzyme, IUBMB systematic classification EC 1.3.1.9. (International Union of Biochemistry and Molecular Biology, www.chem.qmul.ac.uk/iubmb/).

An “effective amount” of a compound of the disclosed invention is the quantity which, when administered to a subject in need of treatment, improves the prognosis of the subject, e.g., delays the onset of and/or reduces the severity of one or more of the subject's symptoms associated with a bacterial infection. The amount of the disclosed compound to be administered to a subject will depend on the particular disease, the mode of administration, and the characteristics of the subject, such as general health, other diseases, age, sex, genotype, body weight and tolerance to drugs. The skilled artisan will be able to determine appropriate dosages depending on these and other factors. Effective amounts of the disclosed compounds typically range between about 0.01 mg/kg per day and about 100 mg/kg per day, and preferably between 0.1 mg/kg per day and about 10 mg/kg/day.

A “pharmaceutically acceptable salt” of the disclosed compound can be used in the disclosed methods. For example, an acid salt of a compound containing an amine or other basic group can be obtained by reacting the compound with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid.

Salts of compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl-β-phenethylamine, dehydroabietylamine, N,N′-bisdehydroabietylamine, glucamine, N-methylglucamine, collidine, quinine, quinoline, and basic amino acid such as lysine and arginine.

A “pharmaceutical composition” is a formulation containing the disclosed compounds, in a form suitable for administration to a subject. The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of active ingredient (i.e., a formulation of the disclosed compound or salts thereof) in a unit dose of composition is an effective amount and may be varied according to the particular treatment involved. It may be appreciated that it may be necessary to make routine variations to the dosage depending on the age and condition of the patient. The dosage will also depend on the route of administration which may be by a variety of routes including oral, pulmonary, rectal, transdermal, subcutaneous, intravenous, intramuscular, intraperitoneal and intranasal.

The compounds described herein, and the pharmaceutically acceptable salts thereof can be used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein. Techniques for formulation and administration of the compounds of the instant invention can be found in Remington: the Science and Practice of Pharmacy, 19^{th} edition, Mack Publishing Co., Easton, Pa. (1995).

For oral administration, the disclosed compounds or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like.

For parental administration of the disclosed compounds, or salts thereof, can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations previously described, the compounds may also be formulated as a depot preparation. Suitable formulations of this type include biocompatible and biodegradable polymeric hydrogel formulations using crosslinked or water insoluble polysaccharide formulations, polymerizable polyethylene oxide formulations, impregnated membranes, and the like. Such long acting formulations may be administered by implantation or transcutaneous delivery (for example subcutaneously or intramuscularly), intramuscular injection or a transdermal patch. Preferably, they are implanted in, or applied to, the microenvironment of an affected organ or tissue, for example, a membrane impregnated with the disclosed compound can be applied to an open wound or burn injury. Thus, for example, the compounds may be formulated with suitable polymeric or hydrophobic materials, for example, as an emulsion in an acceptable oil, or ion exchange resins, or as sparingly soluble derivatives, for example, as a sparingly soluble salt.

In addition to the formulations described previously, the compounds may also be formulated as a topical preparation. Suitable formulations of this type include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administer to a wound site.

In addition to the formulations described previously, the compounds may also be formulated to deliver the active agent by pulmonary means, e.g., administration of an aerosol formulation containing the active agent from, for example, a manual pump spray, nebulizer or pressurized metered-dose inhaler. Suitable formulations of this type can also include other agents, such as antistatic agents, to maintain the disclosed compounds as effective aerosols.

The term “pulmonary” as used herein refers to any part, tissue or organ whose primary function is gas exchange with the external environment, i.e., O₂/CO₂ exchange, within a patient. “Pulmonary” typically refers to the tissues of the respiratory tract. Thus, the phrase “pulmonary administration” refers to administering the formulations described herein to any part, tissue or organ whose primary function is gas exchange with the external environment (e.g., mouth, nose, pharynx, oropharynx, laryngopharynx, larynx, trachea, carina, bronchi, bronchioles, alveoli). For purposes of the present invention, “pulmonary” is also meant to include a tissue or cavity that is contingent to the respiratory tract, in particular, the sinuses.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

In addition to the formulations described above, a formulation can optionally include one or more additional drugs, e.g., other antibiotics, anti-inflammatories, anti-fungals, steroids, decongestants, bronchodialators, and the like. For example, an anti-inflammatory drug or steroid can be co-administered such as ibuprofen, prednisone (corticosteroid) or pentoxifylline.

The term “aryl” group, (e.g., the aryl groups represented by R1 and R2) refers to carbocyclic aromatic groups such as phenyl, naphthyl, and anthracyl. The term “heteroaryl” group (e.g., the heteroaromatic groups represented by R1 and R2) refers to heteroaromatic groups such as imidazolyl, isoimidazolyl, thienyl, furanyl, pyridyl, pyrimidyl, pyranyl, pyrazolyl, pyrrolyl, pyrazinyl, thiazolyl, isothiazolyl, oxazolyl, isooxazolyl, 1,2,3-triazolyl, 1,2,4-triazolyl, and tetrazolyl. As used herein, a “heteroaryl” group is a 5 membered carbocyclic ring containing at least one N, S, or O atom and two double bonds, or a 6 membered carbocyclic ring containing at least one N, S, or O atom and three double bonds.

The term “nonaromatic heterocyclic” (e.g., as represented in X2 by —NR^{e}₂, where N, and the two R^{e} are taken together to form a ring) refers to non-aromatic ring systems typically having four to eight members, preferably five to six, in which one or more ring carbons, preferably one to four, are each replaced by a heteroatom such as N, O, or S. Examples of non-aromatic heterocyclic rings include 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrorolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, and 1-pthalimidinyl.

Some disclosed compounds contain a chiral center. For example, in compound F, the carbon simultaneously attached to the —S—, methyl, and benzoic acid groups is a chiral center. The presence of chiral centers in a molecule gives rise to stereoisomers. For example, a pair of optical isomers, referred to as “enantiomers”, exist for every chiral center in a molecule; and a pair of diastereomers exist for every chiral center in a compound having two or more chiral centers. Where the structural formulas do not explicitly depict stereochemistry, it is to be understood that these formulas encompass enantiomers free from the corresponding optical isomer, racemic mixtures, mixtures enriched in one enantiomer relative to its corresponding optical isomer, a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s).

The term “alkyl” (e.g., the alkyl groups represented by R^{e}), used alone or as part of a larger moiety (e.g., aralkyl, alkoxy, alkylamino, alkylaminocarbonyl, haloalkyl), is a straight or branched non-aromatic hydrocarbon which is completely saturated. Typically, a straight or branched alkyl group has from 1 to about 10 carbon atoms, preferably from 1 to about 4 if not otherwise specified, Examples of suitable straight or branched alkyl group include methyl, ethyl, n-propyl, 2-propyl, n-butyl, sec-butyl, tert-butyl, pentyl, hexyl, heptyl or octyl. A C1-C10 straight or branched alkyl group or a C3-C8 cyclic alkyl group are also referred to as a “lower alkyl” group. An “alkoxy” group refers to an alkyl group that is connected through an intervening oxygen atom, e.g., methoxy, ethoxy, 2-propyloxy, tert-butoxy, 2-butyloxy, 3-pentyloxy, and the like.

The term “aliphatic” includes branched and linear alkyl groups that may contain one or more units of carbon-carbon unsaturation, i.e., carbon-carbon double or triple bonds. A cycloaliphatic group is a cyclic aliphatic group.

The term “cycloalkyl group” is a cyclic alkyl group has from 3 to about 10 carbon atoms, preferably from 3 to about 8. Examples of suitable cycloalkyl groups include cyclopropyl, cyclobutyl, cyclopentyl, cyclohexyl, cycloheptyl and cyclooctyl. A “cycloalkoxy” group refers to a cycloalkyl group that is connected through an intervening oxygen atom, e.g., cyclopentyloxy, cyclohexyloxy, and the like.

An “acyclic” group is a substituent that does not contain a ring. A “monocyclic” group contains only a single ring, for example, a phenyl ring that is not fused to another ring.

Suitable acyclic substituents are those that do not substantially interfere with the pharmaceutical activity of the disclosed compound. A compound or group can have one or more substituents, which can be identical or different. Examples of suitable substituents for a substitutable carbon atom in an alkyl, cycloalkyl, heterocyclic, aromatic, or heteroaromatic group include —OH, halogen (—Br, —Cl, —I and —F), —R, —OR, —CH₂R, —CH₂CH₂R, —OCH₂R, —CH₂OR, —CH₂CH₂OR, —CH₂OC(O)R, —O—COR, —COR, —SR, —SCH₂R, —CH₂SR, —SOR, —SO₂R, —CN, —NO₂, —COOH, —SO₃H, —NH₂, —NHR, —N(R)₂, —COOR, —CH₂COOR, —CH₂CH₂COOR, —CHO, —CONH₂, —CONHR, —CON(R)₂, —NHCOR, —NRCOR, —NHCONH₂, —NHCONRH, —NHCON(R)₂, —NRCONH₂, —NRCONRH, —NRCON(R)₂, —C(═NH)—NH₂, —C(—NH)—NHR, —C(═NH)—N(R)₂, —C(═NR)—NH₂, —C(═NR)—NHR, —C(═NR)—N(R)₂, —NH—C(═NH)—NH₂, —NH—C(═NH)—NHR, —NH—C(═NH)—N(R)₂, —NH—C(═NR)—NH₂, —NH—C(═NR)—NHR, —NH—C(═NR)—N(R)₂, —NRH—C(═NH)—NH₂, —NR—C(═NH)—NHR, —NR—C(═NH)—N(R)₂, —NR—C(═NR)—NH₂, —NR—C(═NR)—NHR, —NR—C(═NR)—N(R)₂, —SO₂NH₂, —SO₂NHR, —SO₂NR₂, —SH, —SOₖR (k is 0, 1 or 2) and —NH—C(═NH)—NH₂. Each R is independently an alkyl, cycloalkyl, benzyl, aromatic, heteroaromatic, or N-anilinyl group that is optionally substituted. Preferably, R is unsubstituted. In addition, —N(R)₂, taken together, can also form a substituted or unsubstituted heterocyclic group, such as pyrrolidinyl, piperidinyl, morpholinyl and thiomorpholinyl. Examples of substituents on group represented by R include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl.

Suitable substituents on the nitrogen of a heterocyclic group or heteroaromatic group include —R′, —N(R′)₂, —C(O)R′, —CO₂R′, —C(O)C(O)R′, —C(O)CH₂C(O)R′, —SO₂R′, —SO₂N(R′)₂, —C(═S)N(R′)₂, —C(═NH)—N(R′)₂, and —NR′SO₂R′. R′ is hydrogen, an alkyl, alkoxy, cycloalkyl, cycloalkoxy, phenyl, phenoxy, benzyl, benzyloxy, heteroaromatic, or heterocyclic group that is optionally substituted. Examples of substituents on the groups represented by R′ include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl. Preferably, R′ is unsubstituted.

An “acidic” group is a group that is substantially deprotonated under physiological conditions. As used herein, “acidic group” includes salts of acidic groups that can be treated to release the acidic group. “Acidic group” also includes carboxylate, sulfate, and sulfonate. As used herein, “acidic group” also includes groups that are bioisosteric for carboxylate, i.e., groups that are recognized by biological systems similarly to carboxylate, for example, sulfonamide, triazole, tetrazole, and the like.

The invention is illustrated by the following examples which are not intended to be limiting in any way.

### EXEMPLIFICATION

The disclosed compounds can be prepared by standard methods starting form appropriate commercially available starting materials.

### Example 1

### Synthesis of fabI Inhibitors of Structural Formula Ia

Compounds represented by structural formula Ia can be prepared by reacting the appropriate 1,3 substituted 2-propenone with 3-cyanopropylthiamide and sodium methoxide in methanol at 65° C. for 12 hours:

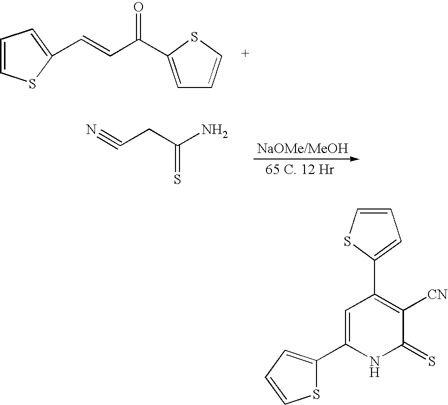

Using this method, compounds Ia-i to Ia-v were prepared:

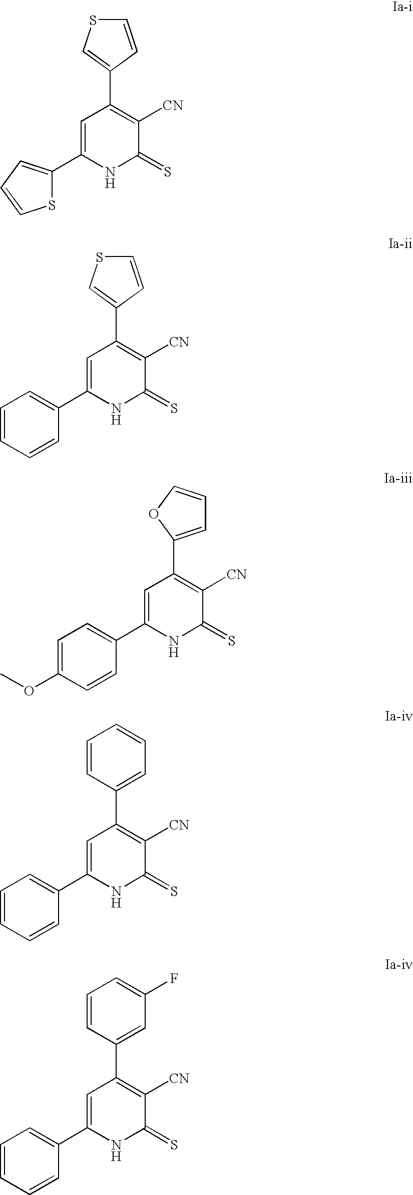

### Example 2

### Synthesis of fabI Inhibitors of Structural Formula Ib

Compounds represented by structural formula Ib can be prepared by reacting the appropriate compound Ia, e.g., as prepared in Example 1, with an alkylating agent. For example, a compound such as Ia-i can be reacted in N,N-dimethylformamide with cesium carbonate and an organo halide such as a substituted benzyl bromide:

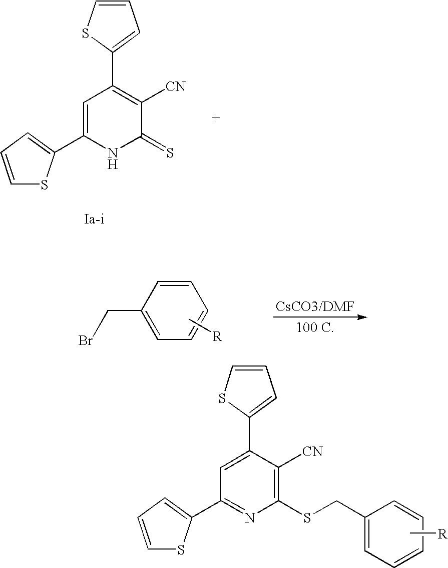

Using this method, compounds represented by structural formulas A-O were prepared.

### Example 3

### fabI Inhibitors Disrupt Fatty Acid Synthesis in Bacteria

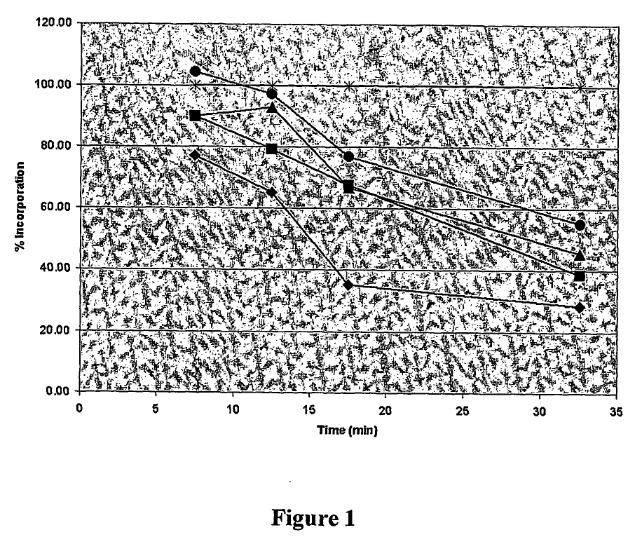

A log-phase culture (OD₆₀₀=0.2) of S. aureus (strain ATCC 700699, American Type Culture Collection, Manassas, Va.) in tryptic soy broth (TSB) medium was added to solutions of the disclosed compounds (0.5 mg/ml stock solution in DMSO) to create solutions of 0, 0.25×, 0.5×, 1×, and 2× minimal inhibitory concentration (MIC). Radioactive (14C) acetic acid was added to 0.4 uCi/ml. After incubation at 37° C. for 0, 5, 10, 15, and 30 minutes, samples were taken, and cells were added to ice cold 10% trichloroacetic acid (TCA) for 30 min. Next, cells were filtered (Millipore MultiScreen-FC filters), washed 2× with 200 ul and 1× with 100 ul cold 10% TCA followed by 2× with 200 ul and 1× with 100 ul cold water. The filters were added to 5 ml scintillation fluid and counts per minute were measured in a scintillation counter. The % inhibition of acetate incorporation into fatty acids was determined by comparison to a control consisting of cells not treated with inhibitor. Incubation with compound A resulted in marked inhibition the activity of fabI as indicated by reduced acetate incorporation into fatty acids, and the inhibition was directly proportional to the concentration of compound A added. FIG. 1 shows the results for 0× (* symbol), 0.25× (circle), 0.5× (triangle), 1× (square), and 2× (diamond).

### Example 4

### fabI Inhibitors have Antibiotic Activity Against Drug-Resistant Bacteria

The sensitivity of methicillin resistant Staphyloccocus aureus (MRSA, strain ATCC 700699, American Type Culture Collection, Manassas, Va.) to the disclosed fabI inhibitors was followed by measuring the minimal inhibitory concentration (MIC) at varying inhibitor levels. This was performed according to NCCLS (National Center for Clinical Laboratory Standards) recommendations (NCCLS, 1997, METHODS FOR DILUTION ANTIMICROBIAL SUSCEPTIBILITY TESTS FOR BACTERIA THAT GROW AEROBICALLY, 4th ed.; approved standard. NCCLS document M7-A4. NCCLS, Wayne, Pa.), in 100 μl volume in 96-well format, with serial two-fold dilutions of drug concentration. The measured MICs for MRSA in μg/mL are shown in Table 1.

TABLE 1

| | | | | | MIC-MRSA | IC50, |
|---|---|---|---|---|---|---|
| Compound | Core | R1 | R2 | R4 | activity, μg/mL | μM |
| A | Ib | R1^{a} | R2^{a} | R4^{h} | 2 | 4 |
| B | Ib | R1^{a} | R2^{c} | R4^{g} | >64 | 12 |
| C | Ib | R1^{a} | R2^{a} | R4^{j} | 32 | 12.3 |
| D | Ib | R1^{a} | R2^{c} | R4ⁱ | >64 | 9.2 |
| E | Ib | R1^{a} | R2^{a} | R4^{a} | 0.75 | 3 |
| F | Ib | R1^{a} | R2^{a} | R4^{b} | 0.75 | 3 |
| G | Ib | R1^{a} | R2^{a} | R4^{c} | 1.5 | 6 |
| H | Ib | R1^{a} | R2^{a} | R4^{d} | 4 | 25 |
| I | Ib | R1^{c} | R2^{e} | R4^{h} | 8 | 25 |
| J | Ib | R1^{a} | R2^{a} | R4^{e} | >64 | 70 |
| K | Ib | R1^{a} | R2^{e} | R4^{e} | >64 | 17 |
| L | Ib | R1^{a} | R2^{c} | R4^{e} | >64 | 18 |
| M | Ib | R1^{a} | R2^{d} | R4^{e} | — | 37 |
| N | Ib | R1^{a} | R2^{d} | R4^{g} | >64 | 46 |
| O | Ib | R1^{b} | R2^{a} | R4^{f} | — | 17 |
| P | Ib | R1^{a} | R2^{a} | R4^{k} | — | 87 |
| Q | Ib | R1^{a} | R2^{d} | R4^{l} | >64 | 33 |

### Example 5

### Antibacterial Activity of Disclosed Compounds is Due to fabI Inhibition

In general, a bacterial strain which is under-producing the target of interest will be more sensitive to test compounds than a wild-type strain if the compound is acting via inhibition of that target. Accordingly, a strain of Staphylococcus aureus that under-expresses a fabI gene under control of a regulated promoter was constructed.

First, the endogenous S. aureus fabI gene was replaced with a drug-resistance marker (erythromycin-resistance, erm^{r}) in a two-step allele-replacement procedure (see F. Fan et al., Plasmid, 2001, 46: 71-75). About 1 Kb of DNA sequence flanking each side of the S. aureus fabI gene (SwissProt Q9RMI3; M. Kuroda et al., Lancet 2001, 357:1225-1240) was PCR-amplified from genomic DNA, ligated to an erythromycin-resistance cassette by cross-over PCR (A. J. Link et al., J. Bacteriol., 1997, 179: 6228-6237), and cloned into an allele-replacement suicide vector carrying a kanamycin-resistance marker (kan^{r}) and the sacB gene encoding levansucrase. This allele-replacement plasmid was introduced by electroporation into an S. aureus host strain carrying the E. coli lactose operon repressor (lacI) and a lac-promoter driving T7 RNA polymerase production from the chromosome. Selection for kan^{r} yielded cells containing the entire plasmid by means of a single cross-over recombination.

Next, the Staphylococcus epidermidis fabI gene (see U.S. Pat. No. 6,380,370) was cloned by PCR and placed under control of the T7 RNA polymerase promoter on a replicating vector carrying a chloramphenicol-resistance marker (cm^{r}). This plasmid was introduced into the S. aureus host strain containing the single cross-over allele-replacement construct. Finally, growth in sucrose and in the presence of the lac promoter inducer isopropyl-beta-D-thiogalactopyranoside (IPTG) was used to select for cells which had completed a second cross-over event to eliminate the sacB gene and produce a total replacement of the endogenous fabI gene with the erm^{r} cassette. The survival of the resulting cells is dependent on production of enoyl reductase from the S. epidermidis fabI gene under IPTG regulation.

The specificity and sensitivity of the resultant strain to added fabI inhibitor A was followed by measuring the minimal inhibitory concentration (MIC) at varying inducer levels. This was performed according to the NCCLS recommendations (op. cit.), in 100 μl volume in 96-well format, with serial two-fold dilutions of drug concentration.

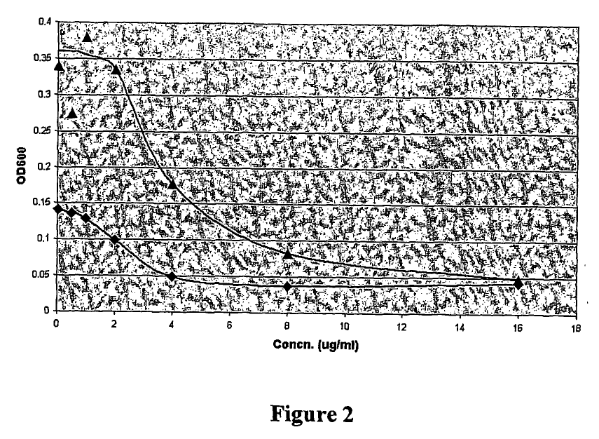

In cells in which fabI is regulated by IPTG, the minimum inhibitory concentration (MIC) of fabI inhibitor A decreased from 8 ug/ml to 4 ug/ml when the inducer (IPTG) level was reduced. FIG. 2 shows the results for the MIC control strain (triangles) and the fabI under-expressing strain (diamonds). The sensitivity of the same strain to a compound which does not inhibit fabI was unchanged. These results demonstrate that the antibiotic activity of fabI inhibitor A is due to inhibition of the fabI enzyme in these bacterial cells.

### Example 6

### Kinetic Assay of fabI Inhibitors

In order to perform more detailed kinetic assays on the disclosed compounds, additional purifed fabI was needed. The E. coli fabI gene was cloned into the pET30a expression vector (Novagen, Inc., Madison, Wis.) and expressed in E. coli BL21(DE3) cells. The purification procedure utilized chromatography with Q-sepharose, blue resin, and HA resin as follows.

Each cell pellet was suspended in a 4 fold-volume of lysis buffer (50 mM KH₂PO₄ pH 8.0, 100 mM NaCl, 2 mM ethylene glycol tetra-acetic acid (EGTA), and 10% glycerol). Cells were broken by passage through a Microfludics cell disrupter 4 times, and the cell lysate was centrifuged at 30,000×g for 20 minutes. The supernatant was applied to a Q-sepharose column pre-equilibrated in a buffer composed of 10 mM Tris-HCl pH 8.0, 0.1 mM EGTA, 1 mM phenylmethylsulfonylfluoride (PMSF), 100 mM NaCl, 10% glycerol, 0.1% β-mercaptoethanol, and 0.02% Brij 35. fabI was eluted with a NaCl gradient (0.1-1M) in the equilibration buffer. The major peak fractions were pooled and concentrated, then dialyzed overnight at 4° C. in 2 L of dialysis buffer (10 mM Tris-HCl pH 7.5, 0.1 mM EGTA, 0.1 mM PMSF, 10% glycerol, 0.1% β-mercaptoethanol, and 0.02% Brij 35). The dialyzed solution was centrifuged at 3,000×g for 20 minutes, and the supernatant was loaded on a blue resin column pre-equilibrated in a buffer composed of 10 mM Tris-HCl pH 7.5, 0.1 mM EGTA, 1 mM PMSF, 50 mM NaCl, 10% glycerol, 0.1% β-mercaptoethanol, and 0.02% Brij 35. fabI was eluted with the equilibration buffer containing NaCl (gradient 50˜1000 mM). The major peak fractions were pooled and dialyzed overnight at 4° C. in 2 L of the equilibration buffer as described above. Then the dialyzed solution was centrifuged at 3,000×g for 20 minutes, and the supernatant was further purified on a hydroxyapatite column pre-equilibrated in a buffer consisting of 20 mM KH₂PO₄ pH 8.0, 0.1 mM EGTA, 0.1 mM PMSF, 10% glycerol, 0.1% β-mercaptoethanol, and 0.02% Brij 35. fabI was eluted with a gradient of KH₂PO₄ up to 500 mM. The peak fractions were pooled and dialyzed in storage buffer (10 mM MOPS pH7.0, 150 mM NaCl, 0.1 mM EGTA, 50% glycerol, 0.02% Brij 35), then stored at −20° C.

The kinetic assay was used to confirm the activity of compounds identified by the single-point assay and screen and to measure the IC50 of compounds which inhibit fabI. It was performed in a 96 well microtiter dish. The final concentrations of each component in each 50 μl reaction are as follows—sodium phosphate pH 7.5, 100 mM; NADH, 200 uM; dithiothreitol (DTT), 1 mM; fabI, 3 ug/ml; crotonoyl-CoA, 0.8 mM. Table 1 shows the IC50 values for compounds A-H.

A 2 ul volume of each compound to be tested was loaded into each well, followed by 30 ul Buffer A (sodium phosphate pH7.5, 100 mM; DTT, 1 mM; fabI, 5 ug/ml, obtained as described below). For IC50 measurements, the compound was tested in serial dilution. The reaction was initiated by adding 20 ul Buffer B (sodium phosphate pH7.5, 100 mM; NADH, 500 uM; DTT, 1 mM; and the substrate, crotonoyl-CoA, 2 mM). The reaction was monitored for 5.5 minutes on a spectrometer by measuring absorption at 340 nm. The rate of reduction of NADH in each reaction well was collected and the percentage inhibition was calculated by using SOFTmax® PRO software (Molecular Devices, Sunnyvale, Calif.). The percentage of inhibition of each compound was calculated according to following formula: Percentage Inhibition=100*(rate in the presence of compound-rate of negative control)/(rate of positive control-rate of negative control)

The negative control was the reaction of fabI in the presence of 2% DMSO but no inhibitory compounds, and the positive control was the mixture of the all reaction components excluding fabI.

While this invention has been particularly shown and described with references to preferred embodiments thereof, it will be understood by those skilled in the art that various changes in form and details may be made therein without departing from the scope of the invention encompassed by the appended claims.

1. A method of treating a subject for a bacterial infection, comprising the step of administering to the subject an effective amount of:
i) a compound represented by the following structural formula, or a pharmaceutically acceptable salt thereof: 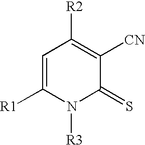
wherein
R1 and R2 are independently monocyclic aryl or heteroaryl groups, wherein the groups represented by R1 and R2 are optionally substituted with triazole, tetrazole, or one or more acyclic substituents provided that R1 is not thienyl when R2 is alkoxy-substituted phenyl;
R3 is —H or an optionally substituted C1-C8 aliphatic, C3-C8 cycloaliphatic, aryl, or heteroaryl group; or
ii) a compound represented by the following structural formula, or a pharmaceutically acceptable salt thereof: 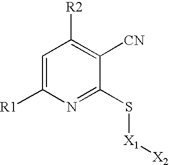
wherein
R1 and R2 are independently monocyclic aryl or heteroaryl groups, wherein the groups represented by R1 and R2 are optionally substituted with triazole, tetrazole, or one or more acyclic substituents;
X1 is a bond or a C1-C3 alkylene chain that is optionally substituted with a C1-C4 alkyl, triazole, tetrazole, or an acidic group;
X2 is an aryl, heteroaryl or C3-C8 cycloaliphatic ring, wherein the group represented by X2 is optionally substituted with triazole, tetrazole, and/or one or more acyclic substituents;
or X2 is triazole, tetrazole, an acidic group, —(CO)NR^{a}R^{b}, —(C═NH)NR^{a}R^{b}, or —(CS)NR^{a}R^{b}, wherein
R^{a} and R^{b} are independently —H or an optionally substituted group selected from aryl, heteroaryl, C3-C8 cycloaliphatic, and C1-C4 alkyl; or
R^{a} and R^{b}, taken together with the nitrogen to which they are bonded, are an optionally substituted non-aromatic heterocyclic group.

2. The method of claim 1 wherein the subject is human.

3. The method of claim 2 wherein the bacterial infection is from a bacterium that expresses a fabI protein.

4. The method of claim 2 wherein the bacterial infection is from Acinetobacter baumanii, Bacillus anthracis, Citrobacter sp., Escherichia coli, Enterobacter sp., Enterococcus faecalis, Enterococcus faecium, Francisella tularensis, Haemophilus influenzae, Klebsiella sp., Listeria monocytogenes, Moraxella catarrhalis, Mycobacterium tuberculosis, Neisseria meningitidis, Proteus mirabilis, Proteus vulgaris, Pseudomonas aeruginosa, Salmonella sp., Serratia sp., Shigella sp., Stenotrophomonas maltophilia, Staphylococcus aureus, or Staphylococcus epidermidis.

5. The method of claim 3 wherein R1 and R2 are independently selected from optionally substituted phenyl, pyridyl, pyrazinyl, pyrimidyl, triazinyl, thienyl, furanyl, pyrrolyl, pyrazolyl, thiazolyl, isothiazolyl, oxazolyl, and isoxazolyl.

6. The method of claim 5 wherein the groups represented by R1 and R2 are optionally substituted with halogen, —OH, —R^{d}, —OR^{d}, triazole, tetrazole, carboxyl, sulfate, sulfonate, —NO₂, —NH₂, —NHCOR^{d}, CONR^{e}₂, NR^{e}₂, or —SO₂NH₂; wherein
each R^{d} is independently a C1-C4 alkyl optionally substituted with 1, 2, or 3 halogens;
each R^{e} is an independently selected C1-C4 alkyl, or both R^{e}, taken together with the nitrogen atom to which they are bonded, are a 4 to 7 membered non-aromatic heterocyclic group.

7. The method of claim 6 wherein the compound is represented by the following structural formula:

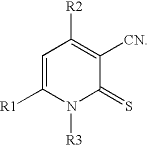

8. The method of claim 7 wherein R1 and R2 are independently selected from optionally substituted phenyl, pyridyl, thienyl, furanyl, and pyrrolyl.

9-11. (canceled)

12. The method of claim 6 wherein the compound is represented by the following structural formula: 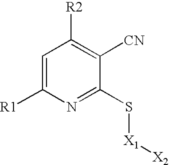
wherein
X1 is a bond or a C1-C3 alkylene chain that is optionally substituted with C1-C4 alkyl, triazole, tetrazole, carboxyl, sulfate or sulfonate; and
X2 is —(CO)NR^{a}R^{b} or an optionally substituted aryl or heteroaryl group.

13. The method of claim 12 wherein X2 is an optionally substituted phenyl, pyridyl, thienyl, furanyl, or pyrrolyl.

14. (canceled)

15. The method of claim 13 wherein
X1 is a C1-C2 alkylene chain optionally substituted with methyl; and
X2 is a phenyl substituted with
a triazole, tetrazole, —CH₂CO₂H, —CH₂CH₂CO₂H, carboxyl, or —NHCOCH₃; and
optionally one or more groups selected from halogen, —R^{d}, —OR^{d}, —NO₂, sulfate, and sulfonate.

16. The method of claim 15 wherein X2 is a phenyl substituted with carboxyl or —NHCOCH₃.

17. The method of claim 12 wherein
X1 is a C1-C2 alkylene chain substituted with triazole, tetrazole, or carboxyl; and
X2 is a phenyl or heteroaryl group optionally substituted with halogen, —R^{d}, —OR^{d}, —NHCOR^{d}, —CONR^{e}₂, triazole, tetrazole, carboxyl, —NO₂, sulfate, or sulfonate.

18. (canceled)

19. The method of claim 12 wherein the compound is represented by the following structural formula: 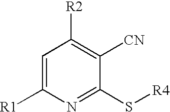
wherein
R1 is selected from structural formulas R1^{a}-R1^{c}: 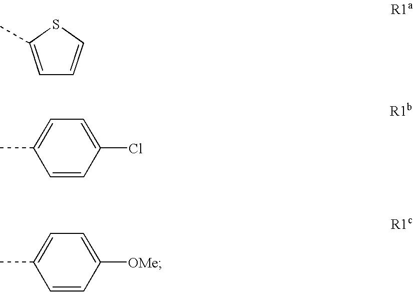
R2 is selected from structural formulas R2^{a}-R2^{e}: 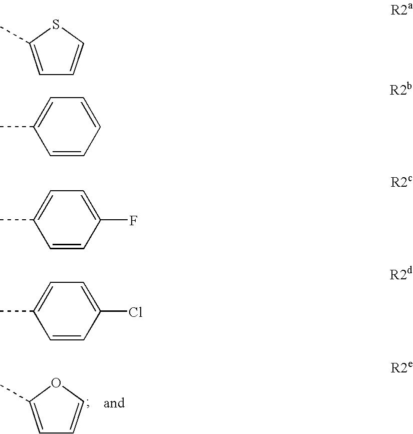
R4 is selected from structural formulas R4^{a}-R4^{l}:

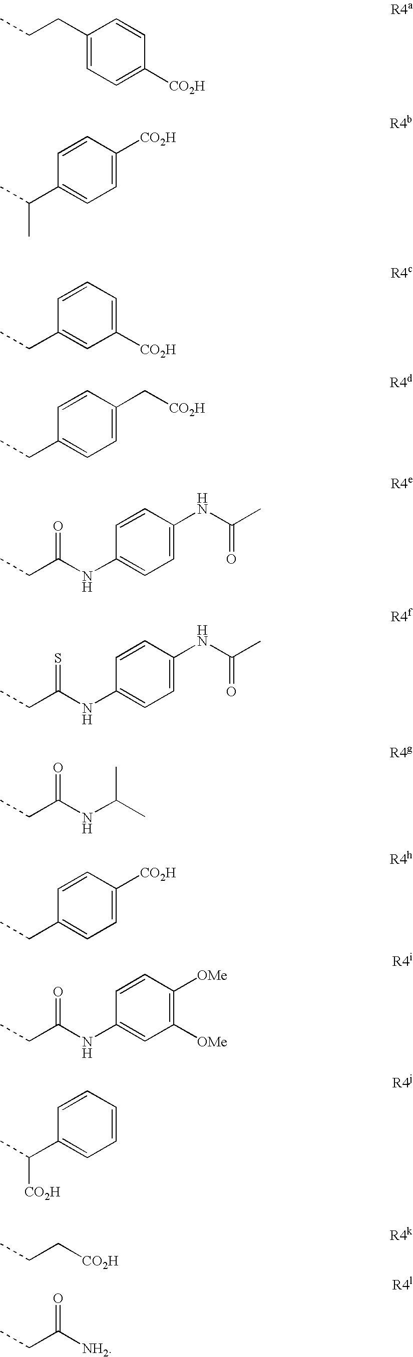

20. (canceled)

21. A compound represented by the following structural formula: 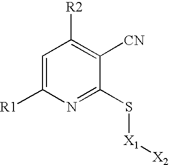
or a pharmaceutically acceptable salt thereof, wherein
R1 and R2 are independently monocyclic aryl or heteroaryl groups, wherein the groups represented by R1 and R2 are optionally substituted with triazole, tetrazole, or one or more acyclic substituents;
X1 is a bond or a C1-C3 alkylene chain that is optionally substituted with a C1-C4 alkyl, triazole, tetrazole, or an acidic group;
X2 is an aryl or heteroaryl ring, wherein the group represented by X2 is optionally substituted with triazole, tetrazole, and/or one or more acyclic substituents;
or X2 is triazole, tetrazole, —(CO)NR^{a}R^{b}, —(C═NH)NR^{a}R^{b}, or —(CS)NR^{a}R^{b}, wherein
R^{a} and R^{b} are independently —H or an optionally substituted group selected from aryl, heteroaryl, and C1-C4 alkyl; provided that both R^{a} and R^{b} are not —H; and
provided that the compound is not represented by one of structural formulas A, B, C, or D:

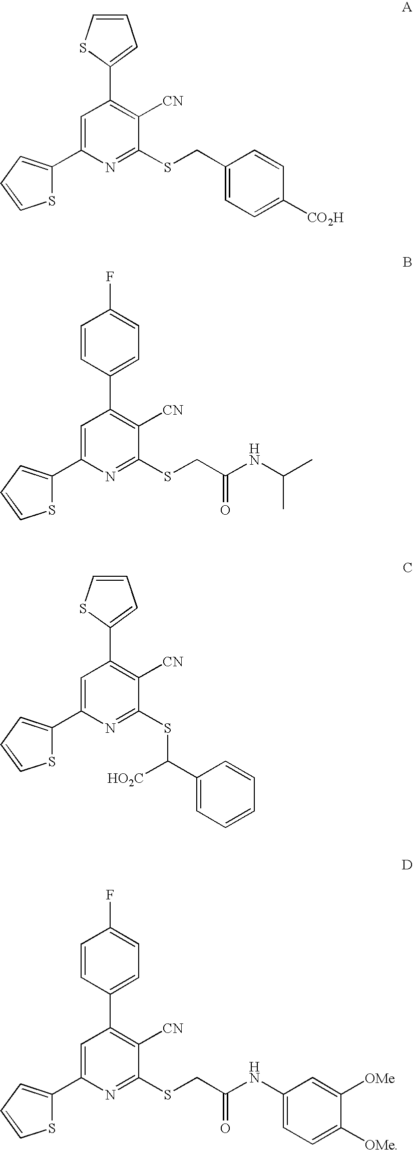

22. The compound of claim 21 wherein R1 and R2 are independently selected from optionally substituted phenyl, pyridyl, pyrazinyl, pyrimidyl, triazinyl, thienyl, furanyl, pyrrolyl, pyrazolyl, thiazolyl, isothiazolyl, oxazolyl, and isoxazolyl.

23. (canceled)

24. The compound of claim 22 wherein:
the groups represented by R1 and R2 are optionally substituted with halogen, —OH, —Rd, —OR^{d}, —NO₂, —NH₂, —NHCOR^{d}, or —SO₂NH₂;
each R^{d} is independently a C1-C4 alkyl optionally substituted with 1, 2, or 3 halogens;
each R^{e} is an independently selected C1-C4 alkyl, or both R^{e}, taken together with the nitrogen atom to which they are bonded, are a 4 to 7 membered non-aromatic heterocyclic group X1 is a bond or a C1-C3 alkylene chain that is optionally substituted with C1-C4 alkyl, triazole tetrazole, —CH₂COOH, —CH₂CH₂COOH, carboxyl, sulfate or sulfonate;
X1 is a bond or a C1-C3 alkylene chain that is optionally substituted with C1-C4 alkyl, triazole, tetrazole, —CH₂COOH, —CH₂CH₂COOH, carboxyl, sulfate or sulfonate; and
X2 is triazole, tetrazole, —(CO)NR^{a}R^{b} or an optionally substituted aryl or heteroaryl group.

25. (canceled)

26. The compound of claim 24 wherein X2 is a phenyl substituted with halogen, —R^{d}, —OR^{d}, —NHCOR^{d}, —CONR^{e}₂, triazole, tetrazole, —CH₂COOH, —CH₂CH₂COOH, carboxyl, —NO₂, sulfate, or sulfonate.

27. The compound of claim 26 wherein
X1 is a C1-C2 alkylene chain optionally substituted with methyl; and
X2 is a phenyl substituted with
a triazole, tetrazole, —CH₂CO₂H, —CH₂CH₂CO₂H, carboxyl, or —NHCOCH₃; and
optionally at least one group selected from halogen, —R^{d}, —OR^{d}, —NO₂, sulfate, and sulfonate.

28. (canceled)

29. The compound of claim 24 wherein
X1 is a C1-C2 alkylene chain substituted with triazole, tetrazole, or carboxyl; and
X2 is a phenyl or heteroaryl group optionally substituted with halogen, —R^{d}, —OR^{d}, —NHCOR^{d}, —CONR^{e}₂, triazole, tetrazole, carboxyl, —NO₂, sulfate, or sulfonate.

30. (canceled)

31. A compound represented by the following structural formula: 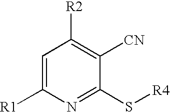
wherein
R1 is selected from structural formulas R1^{a}-R1^{c}: 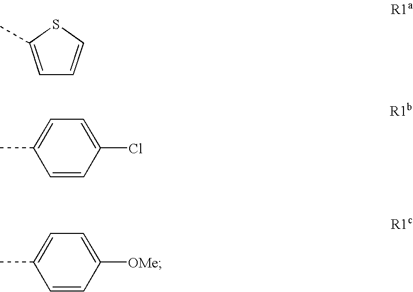
R2 is selected from structural formulas R2^{a}-R2^{e}: 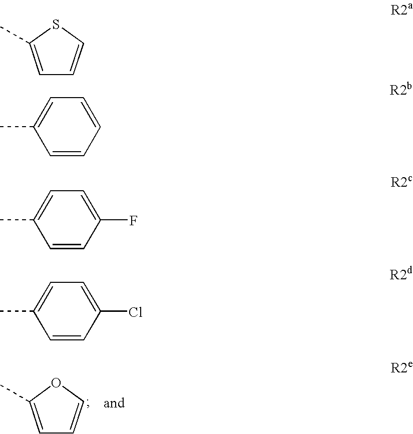
R4 is selected from structural formulas R4^{a}-R4^{f}:

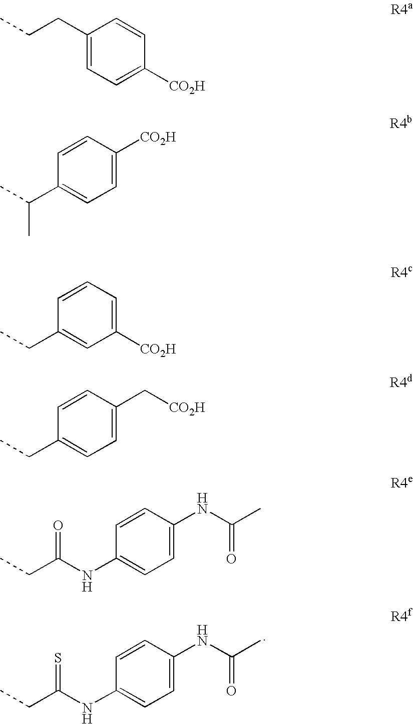

32. The compound of claim 31 wherein
R1 is the group represented by structural formula R1^{b};
R2 is the group represented by structural formula R2^{a}; and
R4 is the group represented by structural formula R4^{f}.

33. (canceled)

34. The compound of claim 31 wherein R1 is the group represented by structural formula R1^{a}, R2 is the group represented by structural formula R2^{a} and R4 is selected from the groups represented by structural formulas R4^{a}-R4^{e}.

35-36. (canceled)

37. The compound of claim 31 wherein
R2 is selected from group represented by structural formulas R₂^{b}, R2^{c}, and R2^{e}; and
R4 is selected from group represented by structural formulas R4^{e} and R4^{g}.

38. A pharmaceutical composition, comprising a pharmaceutically acceptable carrier or diluent and a compound represented by the following structural formula: 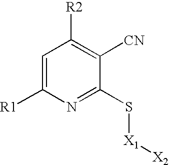
or pharmaceutically acceptable salts thereof, wherein
R1 and R2 are independently monocyclic aryl or heteroaryl groups, wherein the groups represented by R1 and R2 are optionally substituted with triazole, tetrazole, or one or more acyclic substituents;
X1 is a bond or a C1-C3 alkylene chain that is optionally substituted with a C1-C4 alkyl, triazole, tetrazole, or an acidic group;
X2 is an aryl or heteroaryl, wherein the group represented by X2 is optionally substituted with triazole, tetrazole, and/or one or more acyclic substituents;
or X2 is triazole, tetrazole, an acidic group, —(CO)NR^{a}R^{b}, —(C═NH)NR^{a}R^{b}, or —(CS)NR^{a}R^{b}, wherein
R^{a} and R^{b} are independently —H or an optionally substituted group selected from aryl, heteroaryl, and C1-C4 alkyl, provide that if both R^{a} and R^{b} are —H, neither R1 not R2 are furanyl or pyridyl.

39. The composition of claim 38 wherein R1 and R2 are independently selected from optionally substituted phenyl, pyridyl, pyrazinyl, pyrimidyl, triazinyl, thienyl, furanyl, pyrrolyl, pyrazolyl, thiazolyl, isothiazolyl, oxazolyl, and isoxazolyl.

40. (canceled)

41. The composition of claim 39 wherein:
the groups represented by R1 and R2 are optionally substituted with halogen, —OH, —R^{d}, —OR^{d}, —NO₂, —NH₂, —NHCOR^{d}, —CONR^{e}₂, —NR^{e}₂, or —SO₂NH₂;
each R^{d} is independently a C1-C4 alkyl optionally substituted with 1, 2, or 3 halogens;
each R^{e} is an independently selected C1-C4 alkyl, or both R^{e}, taken together with the nitrogen atom to which they are bonded, are a 4 to 7 membered non-aromatic heterocyclic group;
X1 is a bond or a C1-C3 alkylene chain that is optionally substituted with C1-C4 alkyl, triazole, tetrazole, carboxyl, sulfate or sulfonate; and
X2 is triazole, tetrazole, carboxyl, —(CO)NR^{a}R^{b} or an optionally substituted aryl or heteroaryl group.

42. The composition of claim 41 wherein X2 is an optionally substituted phenyl, pyridyl, thienyl, furanyl, or pyrrolyl.

43. (canceled)

44. The composition of claim 42 wherein
X1 is a C1-C2 alkylene chain optionally substituted with methyl; and
X2 is a phenyl substituted with
a triazole, tetrazole, —CH₂CO₂H, —CH₂CH₂CO₂H, carboxyl, or —NHCOCH₃; and
optionally one or more groups selected from halogen, —R^{d}, —OR^{d}, —NO₂, sulfate, and sulfonate.

45. (canceled)

46. The composition of claim 41 wherein
X1 is a C1-C2 alkylene chain substituted with triazole, tetrazole, —CH₂ or carboxyl; and
X2 is a phenyl or heteroaryl group optionally substituted with halogen, —R^{d}, —OR^{d}, —NHCOR^{d}, —CONR^{e}₂, triazole, tetrazole, carboxyl, —NO₂, sulfate, or sulfonate.

47. (canceled)

48. The composition of claim 46 wherein the compound is represented by the following structural formula: 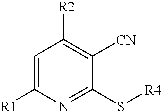
wherein
X2 is an unsubstituted phenyl or heteroaryl group;
R1 is selected from structural formulas R1^{a}-R1^{c}: 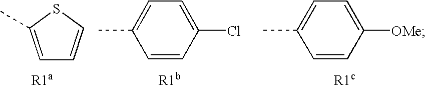
R2 is selected from structural formulas R2^{a}-R2^{e}: 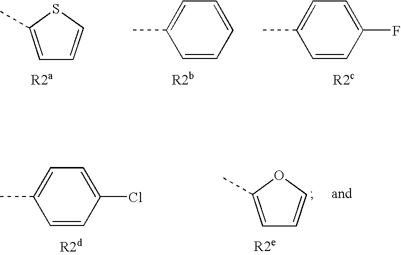
R4 is selected from structural formulas R4^{a}-R4^{j}:

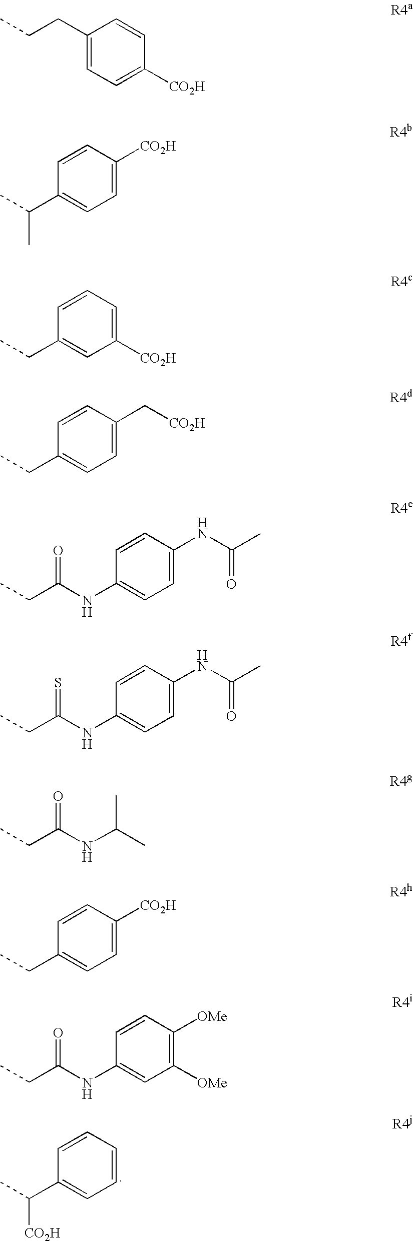

49. The composition of claim 48 wherein the compound is represented by one of structural formulas A to O:

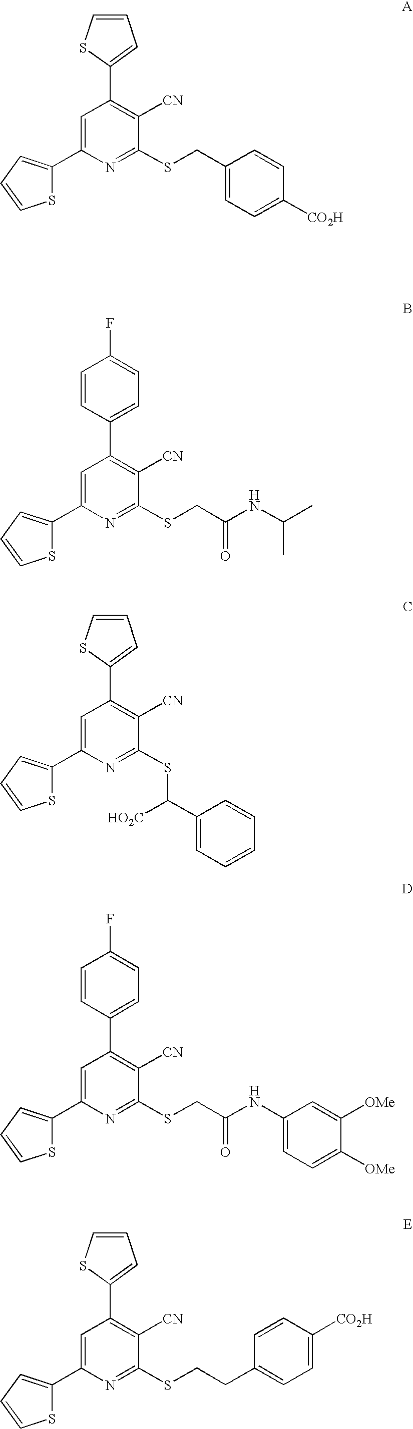

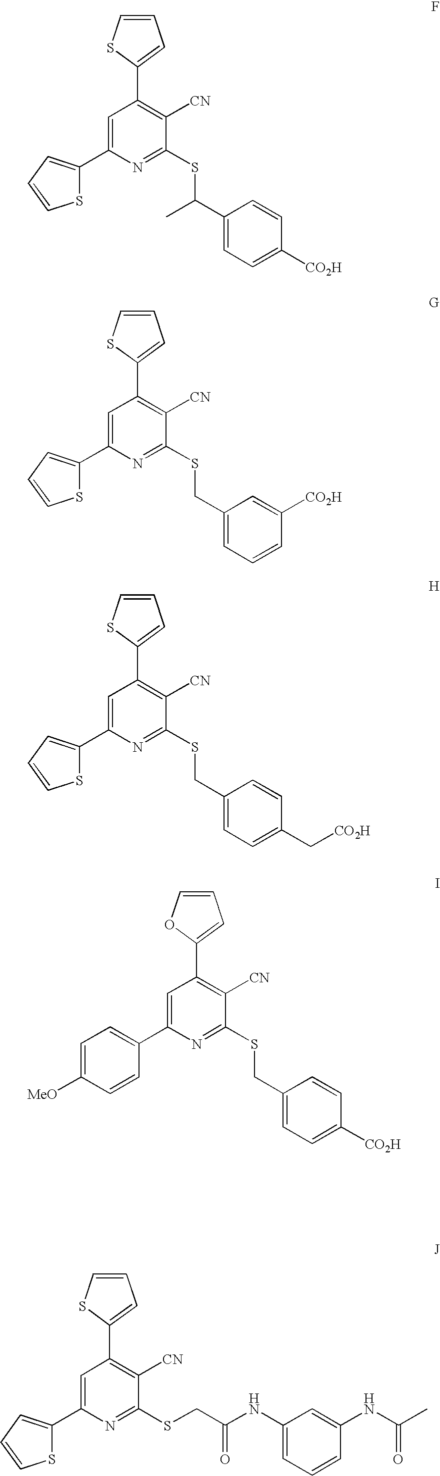

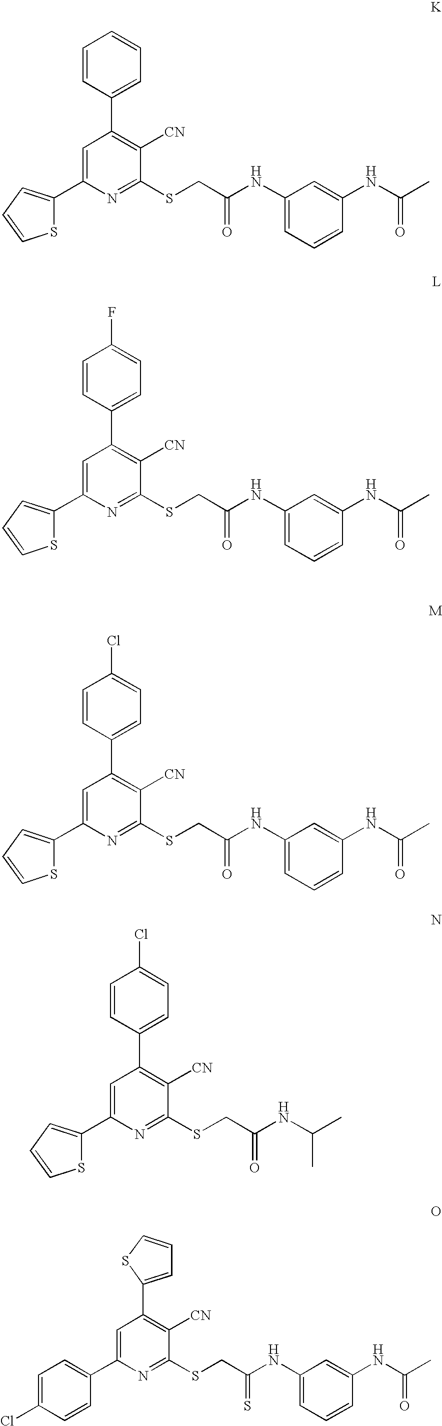

50. (canceled)

51. A compound represented by structural formula Ic: 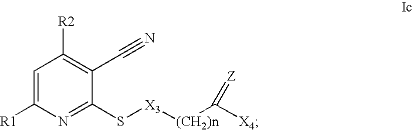
or a pharmaceutically acceptable salt thereof, wherein:
R1 and R2 are independently monocyclic aryl or heteroaryl groups, wherein the groups represented by R1 and R2 are optionally substituted with triazole, tetrazole, or one or more acyclic substituents;
Z is O, S or NR^{f};
X3 is: i) a bond; ii) a C1-C3 alkylene chain that is optionally substituted with a C1-C4 alkyl group or an aromatic group; or iii) a group represented by: 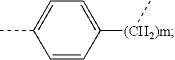
n and m are independently 0 or 1;
X4 is —OH or —NR^{g}R^{h};
R^{f} is H or a C1-C4 alkyl group; and
R^{g} and R^{h} are independently —H or an optionally substituted group selected from: i) aryl that is optionally substituted with one or two C1-C4 alkyl groups, alkoxy groups or acetamido groups; ii) heteroaryl; iii) C3-C8 cycloaliphatic or C1-C6 straight or branched alkyl provided that the compound is not represented by one of structural formulas A, B, C, or D:

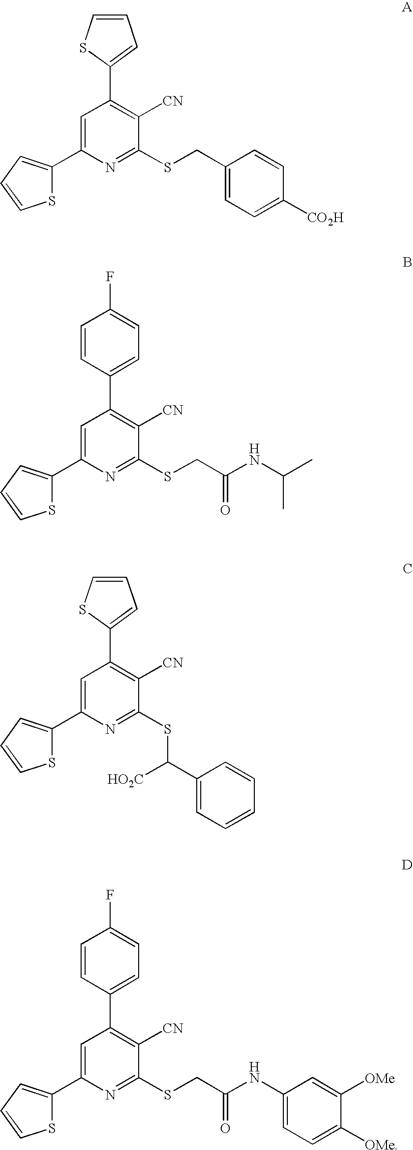

52. (canceled)
